(19) United States
(12) Patent Application Publication
(10) Pub. No.: US 2006/0210633 A1
(43) Pub. Date: Sep. 21, 2006

(54) Programmed drug delivery system

(75) Inventors: Nitin Bhalachandra Dharmadhikari, Mumbai (IN); Yashoraj Rupsinh Zala, Mumbai (IN)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: SUN PHARMACEUTICAL INDUSTRIES LIMITED, Mumbai (IN)

(21) Appl. No.: 10/551,456

(22) PCT Filed: Apr. 5, 2004
(86) PCT No.: PCT/IN04/00092

§ 371 Date: Sep. 30, 2005

(30) Foreign Application Priority Data
Apr. 3, 2003 (IN) 333/MUM/2003
Sep. 29, 2003 (IN) 1021/MUM/2003

(51) Int. Cl. A61K 9/24

(52) U.S. Cl. 424/473

(57) ABSTRACT

The present invention provides a programmed drug delivery system comprising: (a) a core composition comprising one or more beneficial agents and pharmaceutically acceptable excipients, wherein at least one excipient swells when exposed to an aqueous environment, (b) a coat surrounding the core composition, wherein the coat is impermeable to the beneficial agent and other core components, but may be permeable or impermeable to water, (c) passageway in the coat, (d) a composition applied so as to cover the passageway, and optionally, an immediate release composition comprising the same or different beneficial agent.

### FIELD OF THE INVENTION

The present invention relates to a programmed drug delivery system for delivery of a beneficial agent. The versatile and novel drug delivery system is useful for programmed delayed, controlled, spaced or targeted drug delivery. More particularly, the programmed drug delivery system is useful for targeted drug delivery to a specific site in the gastrointestinal tract, at which site the beneficial agent may be delivered as a pulse, or in a rate controlled manner.

### BACKGROUND OF THE INVENTION

Oral administration of a drug provides a plasma level time profile of a drug or its active or inactive metabolite, which can be modulated by the design of the drug delivery system or dosage form. Drug delivery systems releasing the drug slowly over longer duration have been traditionally used to improve therapy by
improving patient compliance to dosage regimens through the decrease in the number of doses the patient has to take in a day, by providing desired effective plasma levels for therapeutic efficacy over the duration of therapy for example throughout the day including at night when the patient is asleep;
decreasing peak plasma levels when they are associated with side effects;
reducing side effects in chronic therapy by reducing the fluctuation in plasma levels seen after multiple dosing of conventional release systems;

when the drug has local action on the gastrointestinal mucosa, to spread the release spatially over the whole of the gastrointestinal mucosa as the drug delivery system is transported in the mucosa by the motility of the gastrointestinal tract.

The drawbacks associated with this mode of delivery are—
Some drugs are absorbed preferentially from a particular region of the gastrointestinal tract.
Some drugs may develop tolerance, i.e. when delivered such that when constant plasma levels are maintained, a decline in pharmacologic response at the constant plasma level of drug is seen. In such instances higher levels are required at a later time.
Drug delivery is not designed according to the chronopharmacokinetics and chronopharmacology of the patient. For example, therapy may optimally require different plasma levels at different times of the day, whereas these systems are not designed to provide such modulated delivery.

For some drugs the maintenance of constant plasma levels over a long duration is unnecessary for therapeutic efficacy. For example, once a peak plasma level is achieved, the desired therapeutic drug action is initiated and persists even if plasma levels decline. In such instance, it is undesirable to maintain the drug plasma levels at the constant higher levels. However, for such drugs there is a need for providing drug delivery systems which will pack multiple doses in a once-daily unit dosage form and deliver each dose at the dosing time as a pulse.

Another traditional mode of release is delayed release utilizing enteric coated dosage form. Enteric coated dosage forms (i.e. dosage forms coated with a polymer having a pH dependent solubility such that it does not dissolve in the gastric fluids but dissolves in the intestinal fluids) have been traditionally used to prevent the release of drug in the stomach and to instead release the drug in the small intestine when—
the drug is unstable in the acidic gastric fluid or undergoes enzymatic degradation in the fluid, or as it passes the gastric mucosa
the drug causes irritation to the gastric mucosa
prolonged duration of drug delivery is desired, which can be given by delivering an initial drug amount from uncoated elements in the dosage form, and delivering the remaining part in the small intestine by enteric coated elements of the dosage form
targeted release of the drug to the small intestine or to the colon is desired
spacing between delivery of a part of the dose immediately in the stomach, and the rest of the dose is desired.

The drawback associated with using enteric coated systems is that the time of emptying of the enteric coated system from the stomach into the small intestine is highly variable and dependent on a variety of physiological factors such as presence or absence of food in the stomach, the type and calories of the food, the physiology of the patient with respect to the gastrointestinal motility and pattern, and the size of the enteric coated unit. Thus, the delay, prolongation, spacing or targeting of drug delivery is not predictably programmed.

### OBJECT OF THE INVENTION

It is an object of the present invention to provide a programmed drug delivery system that is versatile and its various embodiments are suitably designed—
in a first embodiment to deliver a beneficial agent after a programmed predictable delay, which delay is independent of gastric emptying time; this delivery or release being referred to herein as timed release.
in a second embodiment to deliver a beneficial agent after a delay, which delay is dependent on gastric emptying time; this delivery or release being to referred to herein as delayed release.
in a third embodiment to deliver a beneficial agent immediately and after a programmed predictable delay period to provide a time-programmed pulsatile plasma level time profile over short to prolonged durations, which plasma level time profile and the spaced pulses therein are in turn independent of gastric emptying time; this delivery or release being referred to herein as programmed pulsatile delivery.
in a fourth embodiment to deliver a beneficial agent immediately on administration and immediately after delay periods, which delay periods are dependent on gastric emptying time, to provide a pulsatile plasma level time profile over short to prolonged durations, which plasma level time profile and the spaced pulses therein are in turn dependent on gastric emptying time; this delivery or release being referred to herein as pulsatile delivery.
in a fifth embodiment to deliver a beneficial agent immediately on administration and in a controlled manner after delay periods that are independent of gastric emptying time to provide a controlled plasma level time profile over short to prolonged durations, which plasma level time profile and the spaced pulses therein are in turn independent of gastric emptying time; this delivery or release being referred to herein as time-programmed controlled release.
in a sixth embodiment to deliver a beneficial agent immediately on administration, and in a controlled manner after delay periods that are dependent on gastric emptying time to provide a controlled plasma level time profile over short to prolonged durations, which plasma level time profile and the spaced pulses therein are in turn dependent on gastric emptying time; this delivery or release being referred to herein as controlled release.
in an seventh embodiment to deliver a first beneficial agent immediately on administration and to deliver a second beneficial agent immediately after a delay period that is independent of gastric emptying time, to provide spaced pulse delivery of the two beneficial agents, wherein the spacing of the two different beneficial agents is independent of gastric emptying time; this delivery or release being referred to herein as programmed-spaced delivery or release.
in a eighth embodiment to deliver a first beneficial agent immediately on administration and to deliver a second beneficial agent immediately after a delay period that is dependent on gastric emptying time, to provide spaced pulse delivery of the two beneficial agents, wherein the spacing of delivery of the two different beneficial agents is dependent on gastric emptying time; this delivery or release being referred to herein as spaced delivery or release.
in a ninth embodiment to deliver one or more beneficial agents to a target site in the gastrointestinal tract, for example in the upper small intestine or the large intestine, particularly the right colon, where the delivery is a pulse or immediate delivery; this delivery or release being referred to herein as targeted pulse delivery.
in a tenth embodiment to deliver one or more beneficial agents to a target site in the gastrointestinal tract, for example in the upper small intestine or the large intestine, particularly the right colon, in a rate controlled manner; this delivery or release being referred to herein as targeted controlled release.

### SUMMARY OF THE INVENTION

The present invention provides a programmed drug delivery system comprising:
(a) a core composition comprising one or more beneficial agents and pharmaceutically acceptable excipients, wherein at least one excipient swells when exposed to an aqueous environment,
(b) a coat surrounding the core composition, wherein the coat is impermeable to the beneficial agent and other core components, but may be permeable or impermeable to water,
(c) a passageway in the coat,
(d) a composition applied so as to cover the passageway, and
(e) optionally, an immediate release composition comprising the same or different beneficial agent.

### BRIEF DESCRIPTION OF FIGURES

FIG. 1 provides a diagrammatic representation of a “central-band-type” programmed drug delivery system of the present invention, comprising a core (3) with beneficial agent and excipients covered by a coat (2), with the passageway being blocked with a polymer composition. The passageway is present in the center of the system, with the polymer composition being present as a band (1).

FIG. 2 provides a diagrammatic representation of a “off-centered-band-type” programmed drug delivery system of the present invention, comprising a core (3) with beneficial agent and excipients covered by a coat (2), with the passageway being blocked with a polymer composition. The passageway is off-centered and the polymer composition is present as a band (1).

FIG. 3 provides a diagrammatic representation of a “band-on-edge-type” programmed drug delivery system of the present invention, comprising a core (3) with beneficial agent and excipients covered by a coat (2), with the passageway being blocked with a polymer composition. The passageway is present on the edge of the system and the polymer composition is present as a band (1).

FIG. 4 provides a diagrammatic representation of a “central-plug-type” programmed drug delivery system of the present invention, comprising a core (3) with beneficial agent and excipients covered by a coat (2), with the passageway being blocked with a polymer composition. The passageway is present in the center, with the polymer composition being present as a plug (1).

FIG. 5 provides a diagrammatic representation of a “off-centered-plug-type” programmed drug delivery system of the present invention, comprising a core (3) with beneficial agent and excipients covered by a coat (2), with the passageway being blocked with a polymer composition. The passageway is off-centered and the polymer composition is present as a plug (1).

FIG. 6 provides a diagrammatic representation of a “plug-on-edge-type” programmed drug delivery system of the present invention, comprising a core (3) with beneficial agent and excipients covered by a coat (2), with the passageway being blocked with a polymer composition. The passageway is present on the edge of the system and the polymer composition is present as a plug (1).

FIG. 7 provides a diagrammatic representation of a “central-band-type” programmed drug delivery system of the present invention, comprising a core having a first layer (4) with beneficial agent and excipients, and a second layer (5) comprising pharmaceutically acceptable excipients, and optionally a beneficial agent, the core being covered by a coat (2). The passageway in the coat is blocked with a polymer composition. The passageway is present in the center of the system, with the polymer composition being present as a band (1).

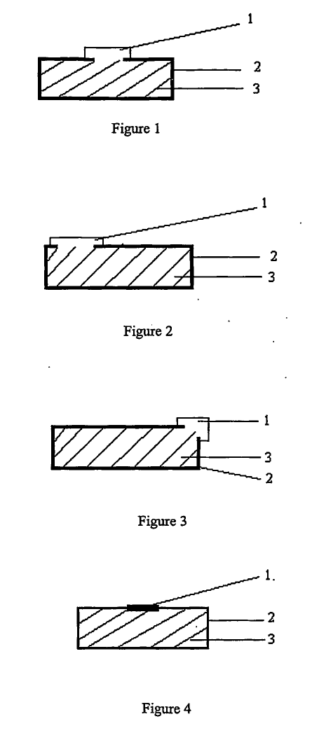

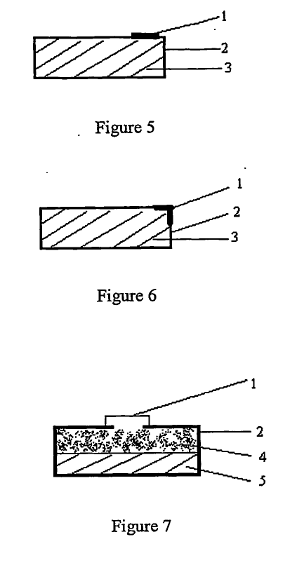

It may be noted that the present invention includes within its scope systems that are similar to those diagrammatically represented in FIG. 7, except that the passageway may be off-centered or may be present on the edge. Also, the polymer composition that covers the passageway may be present in the form of a plug or a band.

### DETAILED DESCRIPTION OF THE INVENTION

The present invention provides a programmed drug delivery system comprising—
(a) a core composition comprising one or more beneficial agents and pharmaceutically acceptable excipients, wherein at least one excipient swells when exposed to an aqueous environment,
(b) a coat surrounding the core composition, wherein the coat is impermeable to the beneficial agent and other core components, but may be permeable or impermeable to water,
(c) a passageway in the coat,
(d) a composition applied so as to cover the passageway, and
(e) optionally, an immediate release composition comprising the same or different beneficial agent.

The core of the programmed drug delivery system of the present invention may be suitably designed to affect a programmed delayed, controlled, spaced or targeted delivery, with a pulsed or controlled release of the one or more beneficial agents.

The present invention also provides a programmed drug delivery system comprising:
(a) a core composition comprising
   (i) a first layer consisting one or more beneficial agents and pharmaceutically acceptable excipients, wherein at least one excipient swells when exposed to an aqueous environment, and
   (ii) a second layer comprising one or more pharmaceutically acceptable excipients selected from water soluble compounds, water insoluble compounds, and a mixture thereof, wherein the second layer may optionally comprise a beneficial agent that is same as or different from that present in the first layer;
(b) a coat surrounding the core composition, wherein the coat is impermeable to the beneficial agent and other core components, but may be permeable or impermeable to water,
(c) a passageway in the coat,
(d) a composition applied so as to cover the passageway, and
(e) optionally, an immediate release composition comprising the same or different beneficial agent.

The programmed drug delivery system of the present invention is useful in providing improved drug delivery. Drugs that may be used in the programmed drug delivery system of the present invention may be selected from the following, viz. alcohol abuse preparations, drugs used for Alzheimer's disease, anesthetics, acromegaly agents, analgesics, antiasthmatics, anticancer agents, anticoagulants and antithrombotic agents, anticonvulsants, antidiabetics antiemetics, antiglaucoma, antihistamines, anti-infective agents, antiparkinsons, antiplatelet agents, antirheumatic agents, antispasmodics and anticholinergic agents, antitussives, carbonic anhydrase inhibitors, cardiovascular agents, cholinesterase inhibitors, treatment of CNS disorders, CNS stimulants, contraceptives, cystic fibrosis management, dopamine receptor agonists, endometriosis management, erectile dysfunction therapy, fertility agents, gastrointestinal agents, immunomodulators and immunosuppressives, memory enhancers, migraine preparations, muscle relaxants, nucleoside analogues, osteoporosis management, parasympathomimetics, prostaglandins, psychotherapeutic agents, sedatives, hypnotics and tranquilizers, drugs used for skin ailments, steroids and hormones

Examples of alcohol abuse preparations are chlorazepate, chlordiazepoxide, diazepam, disulfiram, hydroxyzine, naltrexone and their salts.

Examples of analgesics are acetaminophen, aspirin, bupivacain, buprenorphine, butorphanol, celecoxib, clofenadol, choline, clonidine, codeine, diflunisal, dihydrocodeine, dihydroergotamine, dihydromorphine, ethylmorphine, etodolac, eletriptan, eptazocine, ergotamine, fentanyl, fentoprofen, hyaluronic acid, hydrocodon, hydromorphon, hylan, ibuprofen, lindomethacin, ketorolac, ketotifen, levomethadon, levallorphan, levorphanol, lidocaine, mefenamic acid, meloxicam, meperidine, methadone, morphine, nabumetone, nalbuphin, nefopam, nalorphine, naloxone, naltrexone, naproxen, naratriptan, nefazodone, mormethadon, oxaprozin, oxycodone, oxymorphon, pentazocin, pethidine, phenpyramid, piritramid, piroxicam, propoxyphene, refecoxib, rizatriptan, salsalaketoprofen, sulindac, sumatriptan, tebacon, tilidin, tolmetin, tramadol, zolmitriptan and their salts.

Examples of antiasthmatics are ablukast, azelastine, bunaprolast, cinalukast, cromitrile, cromolyn, enofelast, isamoxole, ketotifen, levcromekalin, lodoxamide, montelukast, ontazolast, oxarbazole, oxatomide, piriprost potassium, pirolate, pobilukast edamine, quazolast, repirinast, ritolukast, sulukast, tetrazolastmeglumine, tiaramide, tibenelast, tomelukast, tranilast, verlukast, verofylline, zarirlukast.

Examples of anticancer agents are adriamycin, aldesleukin, allopurinol, altretamine, amifostine, anastrozole, asparaginase, betamethasone, bexarotene, bicalutamide, bleomycin, busulfan, capecitabine, carboplatin, carmustine, chlorambucil, cisplatin, cladarabine, conjugated estrogen, cortisone, cyclophosphamide, cytarabine, dacarbazine, daunorubicin, dactinomycin, denileukin, dexamethasone, discodermolide, docetaxel, doxorubicin, eloposidem, epirubicin, epoetin, epothilones, estramustine, esterified estrogen, ethinyl estradiol, etoposide, exemestane, flavopirdol, fluconazole, fludarabine, fluorouracil, flutamide, floxuridine, gemcitabine, gemtuzumab, goserelin, hexamethylmelamine, hydrocortisone, hydroxyurea, idarubicin, ifosfamide, interferon, irinotecan, lemiposide, letrozole, leuprolide, levamisole, levothyroxine, lomustine, mechlorethamine, melphalan, mercaptopurine mechlorethamine, megesterol, methotrexate, methylprednisolone, methyltestosterone, mithramycin, mitomycin, mitotane, mitoxantrone, mitozolomide, mutamycin, nilutamide, paclitaxel, pamidronate, pegaspargase, pentostatin, plicamycin, porfimer, prednisolone, procarbazine, rituximab, sargramostim, semustine, streptozocin, tamoxifien, temozolomide, teniposide, testolactone, thioguanine, thiotepa, tomudex, topotecan, toremifene, trastumuzab, tretinoin, semustine, streptozolocin, valrubicin, verteporfin, vinblastine, vincristine, vindesine, vinorelbine and their salts.

Examples of anticoagulants and antithrombic agents are warfarin, dalteparin, heparin, tinzaparin, enoxaparin, danaparoid, abciximab, alprostadil, altiplase, anagralide, anistreplase, argatroban, ataprost, beraprost, camonagreel, cilostazol, clinprost, clopidogrel, cloricromen, dermatan, desirudin, domitroban, drotaverine, epoprostenol, eptifibatide, fradafiban, gabexate, iloprost, isbogrel, lamifiban, lamoteplase, lefradafiban, lepirudin, levosimendan, lexipafant, melagatran, nafagrel, nafamostsat, nizofenone, orbifiban, ozagrel, pamicogrel, parnaparin, quinobendan, reteplase, sarpogralate, satigrel, silteplase, simendan, ticlopidine, vapiprost, tirofiban, xemilofiban, Y20811 and their salts.

Examples of anticonvulsants are carbamazepine, clonazepam, clorazepine, diazepam, divalproex, ethosuximide, ethotion, felbamate, fosphenyloin, gabapentin, lamotrigine, levetiracetam, lorazepam, mephenyloin, mephobaibital, metharbital, methsuximide, oxcarbazepine, phenobarbital, phenyloin, primidone, tiagabine, topiramate, valproic acid, vigabatrin, zonisamide, and their salts.

Examples of antidiabetic agents are acarbose, acetohexamide, carbutamide, chlorpropamide, epalrestat, glibornuride, gliclazide, glimepiride, glipizide, gliquidone, glisoxepid, glyburide, glyhexamide, metformin, miglitol, nateglinide, orlistat, phenbutamide, pioglitazone, repaglinide, rosiglitazone, tolazamide, tolbutamide, tolcyclamide, tolrestat, troglitazone, voglibose and their salts.

Examples of antiemetics are alprazolam benzquinamide, benztropine, betahistine, chlorpromazine, dexamethasone, difenidol, dimenhydrinate, diphenhydramine, dolasetron, domperidone, dronabinol, droperidol, granisetron, haloperidol, lorazepam, meclizine, methylprednisolone, metoclopramide, ondansetron, perphenazine, prochlorperazine, promethazine, scopolamine, tributine, triethylperazine, triflupromazine, trimethobenzamide, tropisetron and their salts.

Examples of antiglaucoma agents are alprenoxime, dapiprazole, dipivefrin, latanoprost, naboctate, pirnabine and their salts.

Examples of antihistamines are acrivastine, activastine, albuterol, azelastine, bitolterol, alimemazine, amlexanox, azelastine, benzydamine, brompheniramine, cetirizine, chlorpheniramine, cimetidine, clemastine, cycloheptazine, cyproheptadine, diclofenac, diphenhydramine, dotarizine, ephedrine, epinastine, epinephrine, ethylnorepinephrine, fenpoterol, fexofenadine, flurbiprofen, hydroxyzine, ibuprofen, isoetharine, isoproterenol, ipratropium bromide, ketorolac, levocetirizine, loratidine, mequitazine, metaproterenol, phenylephrine, phenylpropanolamine, pirbuterol, promethazine, pseudoephedrine, pyrilamine, salmeterol, terbutaline, tranilast, xanthine derivatives, xylometazoline and their salts.

Examples of anti-infective agents are abacavir, albendazole, amantadine, amphotericin, amikacin, aminosalicylic acid, amoxycillin, ampicillin, amprenavir, atovaquin, azithromycin, aztreonam, carbenicillin, cefaclor, cefadroxil, cefamandole, cefazolin, cefdinir, cefepime, cefexime, cefoperazone, cefotaxime, cefotitam, cefoperazone, cefoxitin, cefpodoxine, cefprozil, ceftazidime, ceftibuten, ceftizoxime, ceftriaxone, cefuroxime, cephalexin, chloroquine, cidofovir, cilastatin, ciprofloxacin, clarithromycin, clavulinic acid, clindamycin, colistimethate, dalfopristine, dapsone, daunorubicin, delavirdin, demeclocycline, didanosine, doxycycline, doxorubicin, efavirenz, enoxacin, erythromycin, ethambutol, ethionamide, famsiclovir, fluconazole, flucytocin, foscarnet, fosfomycin, ganciclovir, gatifloxacin, griseofulvin, hydroxychloroquine, imipenem, indinavir, interferon, isoniazide, itraconazole, ivermectin, ketoconazole, lamivudine, levofloxacin, linezolide, lomefloxacin, lovacarbef, mebendazole, mefloquine, meropenem, methanamine, metronidazole, minocycline, moxefloxacin, nalidixic acid, nelfinavir, neomycin, nevirapine, nitrofirantoin, norfloxacin, ofloxacin, olseltamnivir, oxytetracycline, palivizumab, penicillins, perfloxacin, piperacillin, praziquantel, pyrazinamide, pyrimethamine, quinidine, quinupristine, retonavir, ribavirin, rifabutine, rifampicin, rimantadine, saquinavir, sparfloxacin, stavudine, streptomycin, sulfamethoxazole, teramycin, terbinafine, tetracycline, ticarcillin, thiabendazole, tobramycin, trimethoprim, trimetraxate, troleandomycin, trovafloxacin, valacyclovir, vancomycin, zalcitabine, zanamivir, zidovudine and their salts.

Examples of antiparkinsons are amantadine, adrogolide, altinicline, benztropine, biperiden, brasofensine, bromocriptine, budipine, cabergoline, CHF-1301, dihydrexidine, entacapone, etilevodopa, idazoxan, iometopane, lazabemide, melevodopa, carbidopa/levodopa, mofegiline, moxiraprine, pergolide, pramipexole, quinelorane, rasagiline, ropinirole, seligiline, talipexole, tolcapone, trihexyphenidyl and their salts.

Examples of antirheumatic agents are azathiprine, betamethasone, celecoxib, cyclosporin, diclofenac, hydroxychloroquine, indomethacin, infliximab, mercaptobutanedioic acid, methylprednisolone, naproxen, penicillamine, piroxicam, prednisolone, sulfasalazine and their salts.

Examples of platelet agents are abciximab, anagrelide, aspirin, cilostazol, clopidogrel, dipyridamole, epoprostenol, eptifibatide, ticlopidine, tinofiban and their salts.

Examples of antispasmodics and anticholinergic agents are aspirin, atropine, diclofenac, hyoscyamine, mesoprostol, methocarbamol, phenobarbital, scopolamine and their salts.

Examples of antitussives are acetarinophen, acrivastin, albuterol, benzonatate, beractant, brompheniramine, caffeine, calfactant, carbetapentane, chlorpheniramine, codeine, colfuscerin, dextromethorphan, dornase alpha, doxylamine, epinephrine, fexofenadine, guaiphenesin, ipratropium, levalbuterol, metaproterenol, montelukast, pentoxyphyline, phenylephrine, phenylpropanolamine, pirbuterol, poractant alpha, pseudoephedrine, pyrilamine, salbuterol, salmeterol, terbutaline, theophylline, zafirlukast, zileuton and their salts.

Examples of carbonic anhydrase inhibitors are acetazolamide, dichlorphenamide, dorzolamide, methazolamide, sezolamide and their salts.

Examples of cardiovascular agents are abciximab, acebutolol, activase, adenosine, adrenaline, amidarone, amiloride, amlodipine, amyl nitrate, atenolol, atorvastatin, benazepril, bepiridil, betaxalol, bisoprolol, candesartan, captopril, cartenolol, carvedilol, cerivastatin, chlorthalidone, chlorthiazole, clofibrate, clonidine, colestipol, colosevelam, digoxin, diltiazem, disopyramide, dobutamine, dofetilide, doxazosin, enalapril, epoprostenol, eprosartan, esmolol, ethacrynate, erythrityl, felodipine, fenoidapam, fosinopril, fleicainide, flurosemide, fluvastatin, gemfibrozil, hydrochlorthiazide, hydroflumethazine, ibutilide, indapamide, isosorbide, irbesartan, labetolol, lacidipine, lisinopril, losartan, lovastatin, mecamylamine, metoprolol, metaraminol, metazolone, methylchlorthiazide, methyldopa, metyrosine, mexiletine, midrodine, milrinone, moexipril, nadolol, niacin, nicardipine, nicorandil, nifedipine, nimodipine, nisoldipine, nitroglycerin, phenoxybenzamine, perindopril, polythiazide, pravastatin, prazosin, procainamide, propafenone, propranolol, quanfacine, quinapril, quinidine, ranipril, reteplase, simvastatin, sotalol, spironolactone, streptokinase, telmisartan, terazosin, timolol, tocainamide, torsemide, trandolapril, triamterene, trapidil, valsartan and their salts.

Examples of cholinesterase inhibitors are donepezil, edrophonium, neostigmine, pyridostigmine, rivastigmine, tacrine and their salts.

Examples of CNS stimulants are caffeine, doxapram, dexoamphetamine, donepezil, edrophonium, methamphetamine, methylphenidate, modafinil, neostigmine, pemoline, phentermine, pyridostigmine, rivastigmine, tacrin and their salts.

Examples of cystic fibrosis management are dornase alpha, pancrelipase, tobramycin and their salts.

Examples of dopamine receptor agonists are amantadine, cabergoline, fenoldopam, pergolide, pramipexil, ropinirole and their salts.

Examples of drugs used for endometriosis management are danazol, goserelin, leuprolide, nafarelin, norethindrone and their salts.

Examples of drugs used for erectile dysfunction therapy are alprostadil, sildenafil, yohimbine and their salts.

Examples of gastrointestinal agents are aldosetron, bisacodyl, bismuth subsalicylate, celecoxib, difoxin, dipheoxylate, docusate, famotidine, glycopyrrolate, infliximab, lansoprazole, loperamide, metaclopramide, nizatidine, omeprazole, pantoprazole, rabeprazole, ranitidine, simethicone, sucralfate, and their salts.

Examples of immunomodulators and immunosupressives are azathioprin, ceftizoxine, cyclosporin, daclizumab, glatiramer, immunoglobulin, interferon, leflunomide, levamisol, mycophenolate, mausomanab, phthalidomide, ribavirin, sirolimus and their salts.

Examples of drugs used in alzheimer's disease are CP 118954, donepezil, galanthamine, metrifonate, rivastigmine, tacrine, TAK-147 and their salts.

Examples of drugs used for migraine preparations are acetaminophen, dihydroergotamine, divalproex, ergotamine, propranolol, risatriptan, sumatriptan, trimetrexate and their salts.

Examples of muscle relaxants are alcuronium-chloride, azapropazon, atracurium, baclofen, carisoprodol, quinine derivatives, chloromezanon, chlorophenesincarbamate, chlorozoxazon, cyclobenzaprine, dantrolene, decamethoniumbromide, dimethyltubocurariniumchloride, doxacurium, fenyramidol, gallamintriethiodide, guaiphenesin, hexafluoreniumbromide, hexacarbacholinbromide, memantin, mephenesin, meprobamate, metamisol, metaxalone, methocarbamol, mivacurium, orphenadrin, pancuronium, phenazon, phenprobamate, pipecuronium, rapacuronium, rocuronium, succinylcholine, suxamethoniumchloride, tetrazepam, tizanidine, tubocurarine chloride, tybamate, vecuronium and their salts.

Examples of nucleoside analogues are abacavir, acyclovir, didanosine, ganciclovir, gemcitabine, lamivudine, ribavirin, stavudine, zalcitabine and their salts.

Examples of drugs used for osteoporosis management are alendronate, calcitonin, estradiol, estropipate, medroxyprogesterone, norethindrone, norgestimate, pamidronate, raloxifen, risdronate, zolendronate and their salts.

Examples of parasympathomimetics are bethanechol, piperidine, edrophonium, glycopyrolate, hyoscyamine, pilocarpine, tacrine, yohimbine and their salts.

Examples of prostaglandins are alprostadil, epoprostenol, misoprostol and their salts.

Examples of psychotherapeutic agents are acetophenazine, alentemol, alpertine, alprazolam, amitriptyline, aripiprazole, azaperone, batelapine, befipiride, benperidol, benzindopyrine, bimithil, biriperone, brofoxine; bromperidol; bupropion, buspirone, butaclamol, butaperazine; carphenazine, carvotroline, cericlamine, chlorazepine, chlordiazepoxide, chlorpromazine; chlorprothixene, cinperene, cintriamide, citalopram, clomacran, clonazepam, clopenthixol, clopimozide, clopipazan, cloroperone, clothiapine, clothixamide, clozapine; cyclophenazine, dapiprazole, dapoxetine, desipramine, divalproex, dipyridamole, doxepin, droperidol, duloxetine, eltoprazine, eptipirone, etazolate, fenimide, fibanserin, flucindole, flumezapine, fluoxetine, fluphenazine, fluspiperone, fluspirilene, flutrolne, fluvoxamine, gepirone, gevotroline, halopemide, haloperidol, hydroxyzine, hydroxynortriptyline, iloperidone, imidoline, lamotrigine, loxapine, enperone, mazapertine, mephobarbital, meprobamate, mesoridazine, mesoridazine, milnacipran, mirtazapine, metiapine, milenperone, milipertine, molindone, nafadotride, naranol, nefazodone, neflumozide, ocaperidone, odapipam, olanzapine, oxethiazine, oxiperomide, pagoclone, paliperidone, paroxitene, penfluridol, pentiapine perphenazine, phenelzine, pimozide, pinoxepin, pipamperone, piperacetazine, pipotiazine, piquindone, pirlindole, pivagabine, pramipexole, prochlorperazine, prochlorperazine, promazine, quetiapine, reboxetine, remoxipride, remoxipride, risperidone, rimcazole, robolzotan, selegiline, seperidol, sertraline, sertindole; seteptiline, setoperone, spiperone, sunipitron, tepirindole, thioridazine, thiothixene, tiapride, tioperidone, tiospirone, topiramate, tranylcypromine, trifluoperazine, trifluperidol, triflupromazine, triflupromazine, trimipramine, venlafaxine, ziprasidone and their salts.

Examples of sedatives, hypnotics and tranquilisers are bromazepam, buspirone, clazolam, clobazam, chlorazepate, diazepam, demoxepam, dexmedetomitine, diphenyhydramine, doxylamine, enciprazine, estrazolam, hydroxyzine, ketazolam, lorazatone, lorazepam, loxapine, medazepam, meperidine, methobarbital, midazolam, nabilone, nisobamate, oxazepam, pentobarbital, promethazine, propofol, triazolam, zaleplon, zolpidem and their salts.

Examples of drugs used for treatment of skin ailments are acitretin, alclometasone, allitretinoin, betamethasone, calciprotrine, chlorhexidine, clobetasol, clocortolone, clotriamozole, collagenase, cyclosporin, desonide, difluorosone, doxepine, eflornithine, finasteride, fluocinolone, flurandrenolide, fluticasone, halobetasol, hydrochloroquine, hydroquinone, hydroxyzine, ketoconazole, mafenide, malathion, menobenzone, neostigmine, nystatin, podofilox, povidone, tazorotene, tretinoin and their salts.

Examples of steroids and hormones are alclometasone, betamethasone, calcitonin, citrorelix, clobetasol, clocortolone, cortisones, danazol, desmopressin, desonide, desogestrel, desoximetasone, dexamethasone, diflorasone, estradiol, estrogens, estropipate, ethynlestradiol, fluocinolone, flurandrenolide, fluticasone, glucagon, gonadotropin, goserelin, halobetasol, hydrocortisone, leuprolide, levonorgestrel, levothyroxine, medroxyprogesterone, menotropins, methylprednisolone, methyltestosterone, mometasone, naferelin, norditropin, norethindrone, norgestrel, octreolide, oxandrolone, oxymetholone, polytropin, prednicarbate, prednisolone, progesterone, sermorelin, somatropin, stanozolol, testosterone, urofollitropin and their salts.

An embodiment of the programmed drug delivery system of the present invention is particularly useful for agents that are susceptible to the gastric environment such as proton pump inhibitors like pantoprazole, omeprazole, lansoprazole, esomeprazole, rabeprazole, pariprazole, leminoprazole, or an enantiomer, isomer, derivative, free base or salt thereof; lipid-lowering agents such as lovastatin, pravastatin, atorvastatin, simvastatin; agents that are targeted to the intestine for local action such as 5-aminosalicylic acid, corticosteroids such as beclomethasone, budesonide, fluticasone, tixocortol useful in treating Crohn's disease and ulcerative colitis; agents that may be inactivated by the gastric contents such as enzymes like pancreatin, antibiotics such as erythromycin; agents that cause bleeding or irritation of the gastric mucosa such as aspirin, steroids, non-steroidal anti-inflammatory compounds like ibuprofen, naproxen, ketoprofen, fenoprofen, flurbiprofen, oxaprozin, diflunisal, diclofenac, indomethacin, tolmetin, sulindac, etodolac, acetaminophen, platelet inhibitors such as abciximab, intergrelin, dipyridamole; nucleoside analogs such as didanosine, transfer factor preparations, hormones, insulin, and other agents that have decreased stability in the gastric environment, as well as agents that are required for local action in the latter part of the gastrointestinal tract. The agents may be used as their base or as their pharmaceutically acceptable salt or solvate thereof.

The core of the programmed drug delivery system of the present invention may include one or more excipients that are capable of swelling upon exposure to an aqueous environment, and may be selected from hydrophilic non-polymeric compounds and from hydrophilic polymers. The hydrophilic polymers may be of plant, animal, mineral or synthetic origin. The swelling agent may be selected from (A) cellulose derivatives such as C₁₋₄ alkyl celluloses like methyl cellulose and ethyl cellulose; hydroxy C₁₋₄ alkyl celluloses such as hydroxymethyl cellulose, hydroxyethyl cellulose, hydroxypropyl cellulose, and the like; hydroxy C₁₋₄ alkyl C₁₋₄ alkyl celluloses such as hydroxypropyl methylcellulose, hydroxypropyl ethylcellulose and the like; carboxy C₁₋₄ alkyl celluloses such as carboxymethyl cellulose, carboxyethyl cellulose, and their alkali salts; and the like, (B) vinylpyrrolidone polymers such as polyvinyl pyrrolidone, crosslinked polyvinyl pyrrolidone or crospovidone and the like, (C) copolymers of vinyl pyrrolidone and vinyl acetate, (D) gums of plant, animal, mineral or synthetic origin such as (i) agar, alginates, carrageenan, furcellaran obtained from marine sources, (ii) guar gum, gum Arabic, gum tragacanth, karaya gum, locust bean gum obtained from terrestrial plants, (iii) microbial polysaccharides such as dextran, gellan gum, rhamsan gum, welan gum, xanthan gum, and (iv) synthetic or semi-synthetic gums such as propylene glycol alginate, hydroxypropyl guar and modified starches like sodium starch glycolate. The swelling agent used in the present invention may be a combination of the agents mentioned above. Preferably, a combination of two agents is used to provide a controlled swelling thereby causing the coat or core to rupture or burst open at a predetermined time after oral administration of the delivery system. Preferred swelling agents include powdered cellulose, cellulose derivatives, microcrystalline cellulose, silicified microcrystalline cellulose, polyvinylpyrrolidone, crospovidone, sodium starch glycolate, sodium croscarmellose, ion exchange resins and the mixtures thereof. The swelling agent is present in an amount ranging from about 5% to about 95% by weight of the system.

The core of the programmed drug delivery system of the present invention may also include various pharmaceutically acceptable excipients, for example disintegrants such as starch, cellulose derivatives, gums, crosslinked polymers and the like; binders such as starch, gelatin, sugars, cellulose derivatives, polyvinylpyrrolidone and the like; and lubricants such as magnesium stearate, calcium stearate, aluminium stearate, stearic acid, hydrogenated vegetable oils, colloidal silicon dioxide, polyethylene glycol, cellulose derivatives such as carboxyalkyl cellulose and its alkali salts, or mixtures thereof. The pharmaceutically acceptable excipients are used in routine amounts known to a person of skill in the pharmaceutical art.

In one embodiment, the core comprises a first composition comprising one or more beneficial agents, and optionally other pharmaceutically acceptable excipients, and a second composition comprising water soluble compounds for inducing osmosis, and one or more excipients that swell upon imbibing water. Preferably, the first and the second composition are arranged as bilayers, but they may also be admixed with each other. A passageway is formed on the coated core on the side comprising the layer with osmosis-inducing compounds, and it is blocked by a polymer composition that erodes or dissolves at a predetermined time, or upon reaching the latter portion of the gastrointestinal tract. The fluid from the surrounding environment enters the coated core due to osmosis either through the water insoluble coating or the passageway or both, thereby causing the swellable component of the first layer to swell. The pressure exerted by the swollen excipients causes a fracture to develop usually at the passageway and the coat ruptures or bursts, thereby causing opening of the tablets and subsequent release the beneficial agents.

The coat surrounding the core is such that it does not release substantial amount of the drug, until it ruptures or bursts at a predetermined time after oral administration of the delivery system, or at a specific location in the gastrointestinal tract. The coating agents that may be used in the present invention are selected from among water insoluble polymers and hydrophobic compounds known to a person skilled in the art, such that the coat is insoluble in an aqueous environment. The coat is impermeable to the beneficial agent and other core components, but may be permeable or impermeable to water. When it is permeable to water, the core imbibes water from the external environment and swells. When it is impermeable to water, the core absorbs water through the passageway when the polymer composition covering the passageway is eroded or dissolved. The term “impermeable to the beneficial agent” as used herein means that the coat used is such that not more than 15% of the drug is released in one hour from the core to the external environment, when the composition covering the passageway is intact. The core of the system is coated with the coating solution to a weight gain from about 5% to about 15% by weight of the core.

A passageway in the form of an orifice is formed in the coat by suitable means such as mechanical or laser drilling. The orifice may be present in the center of the coated core, may be off-centered or may be present on the edge of the coated core.

The orifice is blocked with a composition, applied such that the beneficial agent is not released until the composition erodes or dissolves. In preferred embodiments, the composition covering the passageway or orifice is made up of polymers that may be pH-independent water soluble polymers and water swellable polymers, or may be pH-dependent polymers, or mixtures thereof. These include, but are not limited to, cellulose and cellulose derivatives such as methyl cellulose, ethyl cellulose, hydroxypropyl cellulose, hydroxymethyl cellulose, hydroxyethyl cellulose, hydroxypropyl methylcellulose, hydroxypropyl methylcellulose acetate succinate, hydroxypropyl methylcellulose phthalate, methacrylic acid and methacrylate esters such as anionic and cationic polymers of methacrylic acid, copolymers of methacrylates, copolymers of acrylates and methacrylates, copolymers of ethacrylate and methylmethacrylate, and mixtures thereof. The polymer composition may block the orifice by forming a band over the orifice or it may plug the orifice. The polymer composition blocks the orifice to provide a release of the one or more beneficial agents from the core at a predetermined time and/or location in the gastrointestinal tract after oral administration. Embodiments that use pH-independent polymers provide release at a predetermined time and those that use pH-dependent polymer provide release at a specific location in the gastrointestinal tract.

The programmed drug delivery system of the present invention may optionally include an immediate release composition comprising the same or different beneficial agent. The immediate release composition may be in the form of granules, pellets, beads, tablets that release the beneficial agent immediately upon oral administration, or it may be present in the form of an immediate release coat or layer partially or wholly covering the programmed drug delivery system.

Pharmaceutically acceptable excipients that may be used in the present invention include water soluble compounds, water insoluble compounds and mixtures thereof. These water soluble and water insoluble compounds include all such compounds listed in “Handbook of pharmaceutical excipients”, 3^{rd} edition, Ed by Arthur Kibbe, Pharmaceutical Press, (2001) and in Remington: The Science and Practice of Pharmacy; edition 19; Mack Publishing Company, Easton, Pa. (1995).

Delayed Release:

One embodiment of the present invention provides a programmed drug delivery system for delayed delivery of the beneficial agent, in the form of a tablet comprising a core comprising one or more beneficial agents, one or more pharmaceutical excipients that swell upon exposure to aqueous medium, and optionally other pharmaceutically acceptable excipients, wherein the core is coated with a water insoluble and water impermeable coating. Aqueous latex dispersions may be used for the purpose and thus the use of organic solvents may be avoided. A passageway is drilled in the coated core, the passageway being blocked with an enteric polymer in the form of a band or a plug. The delay of release in this embodiment is dependent on the gastric emptying time because an enteric polymer is used. When the enteric polymer is applied so as to form a band covering the orifice, then contact between the enteric polymer composition or the enteric band formed thereof and the core is avoided. This is ideally suited for systems where the core is alkaline in nature. For example, drugs from the category of proton-pump inhibitors such as omeprazole, pantoprazole, lansoprazole, esomeprazole and rabeprazole are unstable in an acidic environment and thus are formulated with alkaline excipients, or the drug is used in the form of its alkaline salt for the purpose of achieving the desired stability. An enteric coating over such a composition has two undesirable effects—(a) it tends to dissolve rapidly because of the alkaline milieu of the core, thus releasing the drug in the acidic gastric fluids, defeating the purpose of providing the enteric coat to protect the drug from the acidic gastric fluids, and (b) the acidic enteric coat decreases the pH at the surface of the core, thus decreasing the stability of the drug in that region. Thus, in prior art compositions a subcoat of water soluble or water dispersible excipients is applied. The present invention provides a novel method of total separation of the core from the enteric polymer band by the water insoluble and drug impermeable coat over the core. The impermeable coating does not allow release of the core components in the early portion of the gastrointestinal tract. The pH-dependent polymer composition in the form of a plug or a band blocking the orifice dissolves or erodes upon reaching higher pH environment in the intestine. Water is imbibed by the core through the orifice and the coating when the coating is permeable to water, or through the orifice when the coat is impermeable to water. The swellable component of the core thus swells and exerts a pressure on the coat. A fracture is initiated usually at the orifice, thereby causing the coat to rupture or burst, thereby causing opening of the tablet and subsequent release of the core components in a conventional manner.

According to another embodiment of the present invention, the core of the delayed release system described above further comprises a second layer that ensures complete emptying of the contents of the first layer such that a desirable release is achieved.

Timed Release:

Another embodiment of the present invention provides a programmed drug delivery system that delivers the beneficial agent after a predictable delay, the delay being independent of gastric emptying time, in the form of a tablet comprising a core comprising one or more beneficial agents, one or more pharmaceutical excipients that swell upon exposure to aqueous medium, and optionally other pharmaceutically acceptable excipients, wherein the core is coated with a water insoluble coating. A passageway is drilled in the coat and is covered with a band or a plug of a polymer composition that is soluble or swellable in the gastrointestinal fluids and whose water solubility is pH-independent. Upon erosion or dissolution of the soluble polymer the passageway is exposed and the fluid from the surrounding enters the system, causing it to swell and exert a pressure on the coat. The coat then ruptures to release the contents of the core in a conventional manner. Alternatively, the core may be coated with a polymer composition that is insoluble but permeable to water, and the passageway may be coated with a water-insoluble pH-independent polymer. The water entering the core through the permeable membrane causes the core to swell and the swelling exerts a pressure on the coat. However, the insoluble coating covering the passageway is unaffected by the fluid and the swelling pressure generated inside the system leads to development of a weak point in the coat at the junction of the insoluble coat and the permeable polymer. Hence, the coat ruptures and releases the beneficial agent to the surrounding in a conventional manner.

According to another embodiment of the present invention, the core of the timed release system described above further comprises a second layer that ensures complete emptying of the contents of the first layer such that a desirable release is achieved.

Pulsatile Delivery:

In yet another embodiment the programmed drug delivery system of the present invention provides an immediate delivery of a beneficial agent, followed by a delayed delivery of the same agent, the delay being dependent on gastric emptying time. The system provides a pulsatile plasma level time profile with spaced pulses of the beneficial agent that are dependent on gastric emptying time. At least one delayed release portion is present in the form of a core comprising the beneficial agent, one or more pharmaceutical excipients that swell upon exposure to aqueous medium, and optionally other pharmaceutically acceptable excipients, wherein the core is coated with a water insoluble and water impermeable coating. A passageway is drilled in the coated core and it is blocked with an enteric polymer in the form of a band or a plug. The beneficial agent is released from the core after a delay in a conventional manner, the delay being dependent on gastric emptying time, i.e. after the system reaches the intestine. The immediate release portion may be present in the form of granules, pellets, beads, or tablets, or it may be present as an immediate release coat covering at least a part or whole of the delayed release portion. Alternatively, the immediate release portion may be provided by mixing it with the water insoluble, water impermeable polymer, and using the mixture thus obtained to coat the delayed release core. The system may have more than one delayed release portions, the delayed release portions utilising polymer compositions covering the orifice wherein the polymer used in each delayed release portion dissolves at different pH. Thus, pulsatile release may be provided.

According to another embodiment of the present invention, the core of the pulsatile delivery system described above further comprises a second layer that ensures complete emptying of the contents of the first layer such that a desirable release is achieved.

Programmed Pulsatile Delivery:

Yet another embodiment of the present invention provides a programmed drug delivery system that provides an immediate release of a beneficial agent and a delayed release of the same agent, the delay being independent of gastric emptying time, which release is referred to herein as timed release. The system provides a time-programmed pulsatile plasma level time profile, with spaced pulses of the agent that are independent of the gastric emptying time. At least one timed release portion is present in the form of a core comprising a beneficial agent, one or more pharmaceutical excipients that swell upon exposure to aqueous medium, and optionally other pharmaceutically acceptable excipients, wherein the core is coated with a water insoluble and water impermeable coating. A passageway is drilled in the coat and is covered with a band or a plug of a polymer composition that is soluble or swellable in the gastrointestinal fluids. The beneficial agent is released from the core after a delay in a conventional manner. The immediate release portion may be present in the form of granules, pellets, beads, or tablets, or it may be present as an immediate release coat covering at least a part or whole of the delayed release portion. Alternatively, the immediate release portion may be provided by mixing it with the water insoluble, water impermeable polymer, and using the mixture thus obtained to coat the delayed release core. The system may have more than one timed release portion to provide a pulsatile release, such that pulses of drug are released at times best suited for therapy.

According to another embodiment of the present invention, the core of the programmed pulsatile delivery system described above further comprises a second layer that ensures complete emptying of the contents of the first layer such that a desirable release is achieved.

Controlled Release:

One embodiment of the present invention provides a programmed drug delivery system that provides an immediate release of the beneficial agent, followed by a delayed controlled release of the same agent, the delay being dependent on gastric emptying time. The delayed release portion comprises a core comprising the beneficial agent, one or more pharmaceutical excipients that swell upon exposure to aqueous medium, and optionally one or more pharmaceutically acceptable excipients, the core being surrounded by a coat comprising a polymer composition comprising water insoluble and water impermeable component. A passageway drilled in the wall is covered by a band or a plug of an enteric polymer composition. The enteric polymer erodes or dissolves upon reaching the intestine, thereby exposing the passageway. The fluid from the surrounding environment then enters the system through the passageway and causes swelling of the core and subsequent rupture of the coat. The components of the core and their quantity are selected such that the core delivers the agent in a controlled manner upon rupture of the coat in the intestine. The immediate release portion may be present in the form of granules, pellets, beads, or tablets, or it may be present as an immediate release coat covering at least a part or whole of the delayed release portion. This embodiment uses an immediate release component of a beneficial agent and a delayed controlled release component of the same beneficial agent.

According to another embodiment of the present invention, the core of the controlled release system described above further comprises a second layer that ensures complete emptying of the contents of the first layer such that a desirable release is achieved.

Time-Programmed Controlled Release:

Another embodiment of the present invention comprises a programmed drug delivery system that provides an immediate release of a beneficial agent, followed by a timed controlled release, the delay being independent of gastric emptying time. The delayed release portion of the system comprises a core comprising one or more beneficial agents, one or more pharmaceutical excipients that swell upon exposure to aqueous medium, and optionally other pharmaceutically acceptable excipients, wherein the core is coated with a water insoluble and water impermeable coating. A passageway is drilled in the coat and is covered with a band or a plug of a polymer composition that is soluble in the gastrointestinal fluids. Upon erosion or dissolution of the soluble polymer the passageway is exposed and the fluid from the surrounding enters the system, causing it to swell and exert a pressure on the coat. The coat then ruptures to release the contents of the core in a rate controlled manner. The immediate release portion may be present in the form of granules, pellets, beads, or tablets, or it may be present as an immediate release coat covering at least a part or whole of the delayed release portion. This embodiment uses an immediate release component of a beneficial agent and timed controlled release component of the same beneficial agent.

According to another embodiment of the present invention, the core of the time-programmed controlled release system described above further comprises a second layer that ensures complete emptying of the contents of the first layer such that a desirable release is achieved.

Spaced Delivery:

Another embodiment of the present invention provides a programmed drug delivery system that provides release of a beneficial agent immediately upon oral administration, followed by delayed release of another beneficial agent in a conventional manner, the delay being dependent on gastric emptying time. This embodiment uses an immediate release component of a beneficial agent and a delayed release component of a different beneficial agent.

Programmed Spaced Delivery:

Yet another embodiment of the present invention provides a programmed drug delivery system that provides an immediate release of a beneficial agent upon oral administration, followed by a delayed release of another beneficial agent, the delay being independent of gastric emptying time. This embodiment uses an immediate release component of a beneficial agent and a timed release component of a different beneficial agent.

Targeted Pulse Delivery:

One embodiment of the present invention provides a programmed drug delivery system providing a pulse or immediate delivery of one or more beneficial agents to a targeted site, for example in the upper small intestine or the right colon.

Targeted Controlled Release Delivery:

Yet another embodiment of the present invention provides a programmed drug delivery system that provides targeted delivery of one or more beneficial agents, for example in the upper small intestine or the right colon, in a rate controlled manner.

The programmed drug delivery system of the present invention may be obtained by conventional processes known to a person of skill in the pharmaceutical art. For example, the programmed drug delivery system may be prepared by the conventional process of wet granulation, dry granulation or direct compression. In wet granulation, the beneficial agent along with the various excipients is mixed, granulated, the damp mass screened and dried. The dried mass may be screened, lubricated and compressed to obtain the core of the system. Dry granulation can be done by two processes: (1) slugging, which involves mixing the beneficial agent with excipients, slugging, dry screening, lubrication and compression to obtain the core, and (2) roller compaction process. Direct compression involves compressing the core directly from the powdered material of the beneficial agent and the excipients. The core thus obtained may be coated with the coat using conventional coating techniques. A passageway is then drilled by mechanical or laser drilling, followed by covering of the passageway with a suitable coating composition so as to form a band or a plug.

The following examples merely illustrate the present invention and do not limit the scope of the invention.

### COMPARATIVE EXAMPLE 1

Delayed release tablets comprising omeprazole were prepared as mentioned in Table 1 below.

TABLE 1

| | Quantity | Quantity (Percent |
|---|---|---|
| Ingredients | (mg/tablet) | by weight) |
| Core | | |
| Omeprazole | 20.0 | 8.3 |
| Meglumine | 20.0 | 8.3 |
| Sodium lauryl sulfate (SLS) | 2.4 | 1.0 |
| Polyethylene glycol (PEG 8000) | 2.4 | 1.0 |
| Microcrystalline cellulose | 133.4 | 16.6 |
| (Avicel PH 102) | | |
| Crosslinked polyvinyl pyrrolidone | 40.0 | 55.6 |
| (Crospovidone) | | |
| Mannitol SD 200 | 20.0 | 8.3 |
| Magnesium stearate | 1.8 | 0.75 |

| | |
|---|---|
| Coat | |
| Ethyl cellulose Std 10P | 5% w/w solution coated to a |
| Diethyl phthalate | weight gain of 14-15% by |
| | weight of the core. |
| Enteric band | |
| Hydroxypropyl methylcellulose | 5% w/w solution used to |
| phthalate (HPMCP-50) | coat the orifice |
| Diethyl phthalate | |

Omeprazole, SLS, PEG 8000 and meglumine were mixed together and passed through #40 ASTM (American Society for Materials and Testing) sieve. Avicel PH 102 and crospovidone were mixed with this mixture to obtain a powder blend, which was lubricated with magnesium stearate. The lubricated blend was compressed to obtain a core, which was coated with a solution of ethyl cellulose and diethyl phthalate in organic solvents to a weight gain of about 14 to 15% by weight of the core, using conventional coating methods. An orifice was drilled manually in the center of one side of the tablet and this orifice was covered with an enteric band comprising a solution HPMCP-50 and diethyl phthalate in organic solvents.

The banded tablets thus obtained were subjected to dissolution studies using 900 ml of 0.1N hydrochloric acid for the first two hours and pH 6.8 buffer for an hour. The study was carried out in a United States Pharmacopoeia type II dissolution apparatus at 37° C., at a speed of 100 rpm. The opening time of the tablets, i.e. the time taken by the enteric band to dissolve and the core to swell upon uptake of water through the orifice, was also recorded. These results are mentioned in Table 2 below.

TABLE 2

Percent drug released

| Tablet no. | Opening time | 45 minutes | 60 minutes |
|---|---|---|---|
| 1 | 11 | 67 | 70 |
| 2 | 8 | 64 | 66 |
| 3 | 9 | 59 | 61 |
| 4 | 11 | 59 | 62 |
| 5 | 15 | 63 | 65 |
| 6 | 12 | 61 | 63 |

The tablets were also studied for acid resistance. 24 tablets were used for the study which was carried out using United States Pharmacopoeia type II dissolution apparatus, the medium used being 900 ml of 0.1N hydrochloric acid, and the speed of 100 rpm. No tablet was found to open at the end of 2 hours.

It was observed that the tablets release a maximum of only up to 65% of the drug. This was because the cup formed by the ethyl cellulose coating, upon opening of the tablet, was unable to empty and release the entire core contents. A number of modifications in the composition of the core, changes in the coating polymers and their amounts were carried, but the amount of drug released was found to be no more than about 70%. A change in the tablet shape, weight and thickness also did not address the issue. Hence, a second layer was introduced below the drug-containing layer. A typical embodiment of the present invention is shown in Example 1 below.

### EXAMPLE 1

The programmed drug delivery system of the present invention was obtained as mentioned in Table 3 below.

TABLE 3

| | | Quantity |
|---|---|---|
| | | (Percent by |
| | Quantity | weight of |
| Ingredients | (mg/tablet) | the core) |
| First layer | | |
| Omeprazole | 20.0 | 10.0 |
| Sodium lauryl sulfate (SLS) | 2.0 | 1.0 |
| Disodium hydrogen phosphate, anhydrous | 5.0 | 2.5 |
| Powdered cellulose (Arbocel A300) | 47.0 | 23.5 |
| Crosslinked polyvinyl pyrrolidone | 25.0 | 12.5 |
| (Crospovidone) | | |
| Magnesium stearate | 1.0 | 0.5 |
| Second layer | | |
| Mannitol SD 200 | 76.43 | 38.22 |
| Crosslinked polyvinyl pyrrolidone | 21.43 | 10.72 |
| (Crospovidone) | | |
| Sodium lauryl sulfate (SLS) | 1.00 | 0.5 |
| Magnesium stearate | 0.71 | 0.36 |
| Colour | q.s. | |

| | |
|---|---|
| Coat | |
| Ethyl cellulose Std 10P | 5% w/w solution coated to a |
| Diethyl phthalate | weight gain of 12-14% |
| | by weight of the core. |
| Enteric band | |
| Hydroxypropyl methylcellulose phthalate | 5% w/w solution used to |
| (HPMCP-50) | coat the orifice |
| Diethyl phthalate | |

Omeprazole SLS and disodium hydrogen phosphate are mixed together to obtain a blend, which is passed through ASTM #40. This blend is then mixed with crospovidone and powdered cellulose, which is previously passed through ASTM #40. The blend is lubricated with magnesium stearate to obtain the first layer blend. Mannitol is mixed with crospovidone and colour to obtain a colour mix for the second layer. This is then lubricated with magnesium stearate to obtain the second layer blend. The two blends are compressed by conventional means to obtain a bilayered tablet, which is then coated with the coating solution of ethyl cellulose and diethyl phthalate. An orifice is drilled in the coat on the side of the first layer. This orifice is then covered with the enteric banding composition comprising HPMCP-50 and diethyl phthalate.

The tablets thus obtained were subjected to dissolution test using United States Pharmacopoeia dissolution apparatus, type II, using 900 ml of pH 6.8 buffer at a speed of 100 rpm. The dissolution profile of the tablets is recorded in Table 4 below.

TABLE 4

| Time (minutes) | % drug released |
|---|---|
| 0 | 0 |
| 45 | 86 |
| 60 | 90 |

### EXAMPLE 2

A programmed drug delivery system of the present invention is obtained as mentioned in Table 5 below.

TABLE 5

| | | Quantity |
|---|---|---|
| | Quantity | (Percent by |
| Ingredients | (mg/tablet) | weight) |
| First layer | | |
| Omeprazole | 20.0 | 9.09 |
| Sodium lauryl sulfate (SLS) | 2.0 | 0.9 |
| Disodium hydrogen phosphate, anhydrous | 5.0 | 2.25 |
| Microcrystalline cellulose (Avicel PH200) | 28.0 | 12.77 |
| Powdered cellulose (Arbocel A300) | 29.0 | 13.2 |
| Crosslinked polyvinyl pyrrolidone | 25.0 | 11.36 |
| (Crospovidone) | | |
| Magnesium stearate | 1.0 | 0.4 |
| Second layer | | |
| Dicalcium phosphate (direct compression | 81.4 | 37.0 |
| grade) | | |
| Polyvinyl pyrrolidone (PVP K-30) | 22.0 | 10.0 |
| Disodium hydrogen phosphate, anhydrous | 5.5 | 2.5 |
| Magnesium stearate | 1.1 | 0.5 |
| Colour | q.s. | |

| | |
|---|---|
| Coat | |
| Ethyl cellulose Std 10P | 5% w/w solution coated to a |
| Diethyl phthalate | weight gain of 12-14% |
| | by weight of the core. |
| Enteric band | |
| Hydroxypropyl methylcellulose phthalate | 5% w/w solution used to |
| (HPMCP -50) | coat the orifice |
| Diethyl phthalate | |

The tablets are obtained as per the process mentioned in Example 1 above.

The tablets thus obtained were subjected to dissolution test using United States Pharmacopoeia dissolution apparatus, type II, using 900 ml of pH 6.8 buffer at a speed of 100 rpm. The dissolution profile of the tablets is recorded in Table 6 below.

TABLE 6

| Time (minutes) | % drug released |
|---|---|
| 0 | 0 |
| 45 | 95 |
| 60 | 97 |

Comparing results of the dissolution tests listed in Table 2, Table 4 and Table 6, it is evident that the presence of a second layer ensures a release of about 90% or more at the end of 60 minutes. The comparative example 1 without the second layer was not able to achieve more than 70% of drug release at the end of 60 minutes.

### EXAMPLE 3

The programmed drug delivery system of the present invention was obtained in the form of tablets, wherein lactose was substituted for the drug. The dummy tablets were obtained as mentioned in Table 7 below—

TABLE 7

| Ingredients | Quantity (% w/w) |
|---|---|
| Microcrystalline cellulose (Avicel PH 101) | 56.0 |
| Crosslinked polyvinylpyrrolidone | 15.0 |
| (Crospovidone) | |
| Meglumine | 16.6 |
| Lactose anhydrous (Pharmatose DCL-21) | 6.66 |
| Colloidal silicon dioxide (Aerosil) | 3.0 |
| Magnesium stearate | 1.66 |
| Talc | 1.0 |
| Ethyl cellulose N-10 | Used as a 5% w/w solution |
| Diethyl phthalate | for coating the tablet core |
| Eudragit L-100-55 | Quantity dependent on |
| | weight gain desired |

Microcrystalline cellulose, crospovidone and meglumine were mixed thoroughly and granulated using purified water. The granules were mixed with lactose, colloidal silicon dioxide, magnesium stearate and talc. The mixture thus obtained was compressed using conventional means. The compressed tablets were coated with a 5% coating solution of ethyl cellulose and diethyl phthalate in dichloromethane and methanol, to a weight gain of 3.5%. An orifice was then drilled in the coated tablet, and the orifice was finally covered with a band comprising Eudragit L-100-55 as the enteric polymer, and diethyl phthalate as plasticiser. The amount of weight gain of the tablet after coating with the enteric polymer was varied to achieve different time of opening in pH 6.8 buffer.

The tablets were evaluated for opening time study and were introduced in pH 6.8 buffer. Tablets that were not drilled, as well as drilled tablets not covered by an enteric band were also introduced in pH 6.8 buffer for comparison. Five tablets of each type were included for the study. The observations are recorded in Table 8 below.

TABLE 8

| | | Opening time in |
|---|---|---|
| Tablet no. | Tablet description | pH 6.8 buffer |
| 1 | Undrilled | 1 hour 10 minutes |
| 2 | Undrilled | 1 hour 10 minutes |
| 3 | Undrilled | 1 hour 20 minutes |
| 4 | Undrilled | Not for 2 hours |
| 5 | Undrilled | Not for 2 hours |
| 6 | Drilled | 20 minutes |
| 7 | Drilled | 20 minutes |
| 8 | Drilled | 20 minutes |
| 9 | Drilled | 25 minutes |
| 10 | Drilled | 25 minutes |
| 11 | Enteric banded | 25 minutes (0.9% |
| | | weight gain) |
| 12 | Enteric banded | 30 minutes (0.9% |
| | | weight gain) |
| 13 | Enteric banded | 35 minutes (1.25% |
| | | weight gain) |
| 14 | Enteric banded | 35 minutes (1.27% |
| | | weight gain) |
| 15 | Enteric banded | 1 hour (1.9% |
| | | weight gain) |

As is apparent from the table above, the opening time is predictable and reliable for enteric banded tablets. An increase in weight gain after coating with the enteric polymer results in an increase in opening time of the tablet.

### EXAMPLE 4

The programmed drug delivery system of the present invention may be obtained in the form of tablets as mentioned in Table 9 below. The lactose anhydrous used as per Table 7 above may be substituted by any drug. In this example it was substituted by Esomeprazole magnesium.

TABLE 9

| Ingredients | Quantity (% w/w) |
|---|---|
| Microcrystalline cellulose (Avicel PH 101) | 56.0 |
| Crosslinked polyvinylpyrrolidone | 15.0 |
| (Crospovidone) | |
| Meglumine | 16.66 |
| Esomeprazole magnesium | 6.66 |
| Colloidal silicon dioxide (Aerosil) | 3.0 |
| Magnesium stearate | 1.66 |
| Talc | 1.0 |
| Ethyl cellulose N-10 | Used as a 5% w/w solution |
| Eudragit L-100-55 | for coating the tablet core |

The tablet core was obtained as mentioned in Example 3 above. The compressed cores were coated with a 5% w/w solution of ethyl cellulose and Eudragit L-100-55 in dichloromethane and ethanol, to a weight gain of 6% and 7% in a fluid bed coater.

The tablets were evaluated for opening time study in 0.1N hydrochloric acid and pH 6.8 buffer. Five tablets were used for the study. The observations are recorded in Table 10 below.

TABLE 10

Opening time

| Tablets used | | pH 6.8 |
|---|---|---|
| for the study | 0.1N HCl | buffer |
| Tablets comprising esomeprazole | None of the | 28-30 |
| magnesium as the drug, coated | tablets opened | minutes |
| to a weight gain of 6% by | for 2 hours | |
| weight of the core | | |

The above example demonstrates a programmed drug delivery system for drugs, which system does not release the beneficial agent in the early portion of the gastrointestinal tract, i.e. there is no release for 2 hours after oral administration. However, the drug is released within 28-30 minutes in pH 6.8 buffer indicating that the system is capable of delivering the drug in the latter portion of the gastrointestinal tract in a reliable manner. The core of the system may be suitably designed to affect a conventional, sustained, controlled or pulsed release of the beneficial agent.

### EXAMPLE 5

The programmed drug delivery system of the present invention may be obtained in the form of tablets as mentioned in Table 11 below.

TABLE 11

| Ingredients | Quantity (% w/w) |
|---|---|
| Omeprazole (micronised) | 9.09 |
| Silicified microcrystalline | 75.86 |
| cellulose (Prosolv SMCC 90 ®) | |
| Sodium lauryl sulfate | 1.0 |
| Polyethylene glycol (PEG 4000) | 1.0 |
| Meglumine | 4.5 |
| Crosslinked polyvinyl | 8.0 |
| pyrrolidone (Crospovidone) | |
| Magnesium stearate | 0.5 |
| Ethyl cellulose standard 10 P | Used as a 5% w/w solution |
| Diethyl phthalate | 5% w/w of ethyl cellulose |
| Hydroxypropyl methylcellulose | Used as a 5% w/w solution |
| phthalate (HPMCP-50) | |
| Diethyl phthalate | 10% w/w of HPMCP-50 |

Omeprazole, sodium lauryl sulfate, PEG 4000 and meglumine were mixed together and passed through ASTM (American Society of Testing and Materials) #40 sieve. The mixture thus obtained was mixed with Prosolv and crospovidone to obtain a blend. This blend was lubricated with magnesium stearate and compressed to obtain the core. The core was coated with a solution of ethyl cellulose and diethyl phthalate in a mixture of dichloromethane-methanol (4:1) to a weight gain of from about 14% to about 15% by weight of the core. An orifice was drilled in the coated core and this orifice was coated with a solution of HPMCP-50 and diethyl phthalate in a mixture of dichloromethane-methanol (3:1).

The programmed drug delivery system thus obtained was subjected to tablet opening time studies using United States Pharmacopoeia type II dissolution apparatus at 37° C., at a speed of 75 rpm. The medium used was 900 ml of 0.1N HCl for the first two hours, followed by pH 6.8 buffer for one hour. None of the tablets opened in 0.1N HCl for two hours, while all the tablets opened within 9 minutes and 23 minutes in pH 6.8 buffer. The average opening time in pH 6.8 buffer was 18 minutes.

### EXAMPLE 6

The programmed drug delivery system of the present invention may be obtained in the form of tablets as mentioned in Table 12 below.

TABLE 12

| Ingredients | Quantity (% w/w) |
|---|---|
| Omeprazole (micronised) | 9.09 |
| Silicified microcrystalline | 75.86 |
| cellulose (Prosolv SMCC 90 ®) | |
| Sodium lauryl sulfate | 1.0 |
| Polyethylene glycol (PEG 4000) | 1.0 |
| Disodium hydrogen phosphate, | 0.91 |
| anhydrous | |
| Crosslinked polyvinyl | 8.0 |
| pyrrolidone (Crospovidone) | |
| Magnesium stearate | 0.5 |
| Lactitol monohydrate | 3.6 |
| Ethyl cellulose standard 10 P | Used as a 5% w/w solution |
| Diethyl phthalate | 5% w/w of ethyl cellulose |
| Hydroxypropyl methylcellulose | Used as a 5% w/w solution |
| phthalate (HPMCP-50) | |
| Diethyl phthalate | 10% w/w of HPMCP-50 |

Omeprazole, sodium lauryl sulfate, PEG 4000 and anhydrous disodium hydrogen phosphate were mixed together and passed through ASTM (American Society of Testing and Materials) #40 sieve. The mixture thus obtained was mixed with Prosolv, lactitol monohydrate and crospovidone to obtain a blend. This blend was lubricated with magnesium stearate and compressed to obtain the core. The core was coated with a solution of ethyl cellulose and diethyl phthalate in a mixture of dichloromethane-methanol (4:1) to a weight gain of from about 14% to about 15% by weight of the core. An orifice was drilled in the coated core and this orifice was coated with a solution of HPMCP-50 and diethyl phthalate in a mixture of dichloromethane-methanol (3:1).

The programmed drug delivery system thus obtained was subjected to tablet opening time studies using United States Pharmacopoeia type II dissolution apparatus at 37° C., at a speed of 75 rpm. The medium used was 900 ml of 0.1N HCl for the first two hours, followed by pH 6.8 buffer for one hour. None of the tablets opened in 0.1N HCl for two hours, while all the tablets opened within 9 minutes and 23 minutes in pH 6.8 buffer. The average opening time in pH 6.8 buffer was 18 minutes.

### EXAMPLE 7

The programmed drug delivery system of the present invention may be obtained in the form of bilayered tablets as mentioned in Table 13 below.

TABLE 13

| Ingredients | Quantity mg/tablet) |
|---|---|
| First layer | |
| Omeprazole | 20.0 |
| Disodium hydrogen phosphate | 5.00 |
| Sodium lauryl sulfate | 2.00 |
| Mannitol | 10.00 |
| Powdered cellulose (Arbocel) | 20.00 |
| Microcrystalline cellulose (Avicel) | 47.50 |
| Crospovidone | 14.50 |
| Magnesium stearate | 1.00 |
| Second layer | |
| Powdered cellulose (Arbocel) | 40.00 |
| Microcrystalline cellulose (Avicel PH 112) | 76.00 |
| Crospovidone | 25.00 |
| Disodium hydrogen phosphate | 5.00 |
| Sodium lauryl sulfate | 2.00 |
| Color | 0.50 |
| Magnesium stearate | 1.50 |
| Coat | |
| Ethyl cellulose standard | |
| Diethyl phthalate | |

Omeprazole, disodium hydrogen phosphate, sodium lauryl sulfate and mannitol were mixed together to obtain a dry blend. This was further mixed with Arbocel, microcrystalline cellulose, crospovidone and magnesium stearate to obtain a first blend that will form the first layer. The blend for the second layer was obtained by mixing Arbocel, microcrystalline cellulose, crospovidone, disodium hydrogen phosphate, sodium lauryl sulfate, color and magnesium stearate. The two blends were then compressed to obtain the bilayered tablet.

A 5% solution of ethyl cellulose in dichloromethane and methanol, comprising ethyl cellulose, diethyl phthalate, talc and Tween 80 was prepared, and the pH of the solution was adjusted to about pH 7.5 with sodium hydroxide. This coating solution was used to coat the bilayered tablets to a weight gain of about 10%.

An orifice was drilled in the tablet on the side of the layer comprising the drug. The orifice was coated with a solution of HPMCP-50 and diethyl phthalate in a mixture of dichloromethane-methanol (3:1). It may be noted that the coating solution is applied such that a layer or band or patch covers only the orifice. The coating solution used may be any of the compositions as mentioned in examples herein.

### EXAMPLE 8

The programmed drug delivery system of the present invention may be obtained in the form of tablets as mentioned in Table 14 below.

TABLE 14

| Ingredients | Quantity (% w/w) |
|---|---|
| Intragranular | |
| Mesalamine | 57.14 |
| Silicified microcrystalline | 8.85 |
| cellulose (Prosolv SMCC 90) | |
| Sodium lauryl sulfate (SLS) | 0.85 |
| Polyvinyl pyrrolidone (PVP K30) | 2.0 |
| Extragranular | |
| Silicified microcrystalline | 27.28 |
| cellulose (Prosolv SMCC 90) | |
| Crosslinked polyvinyl | 2.85 |
| pyrrolidone (Crospovidone) | |
| Magnesium stearate | 1.0 |
| Coating | |
| 18% aqueous ethyl cellulose | Coated to a weight gain |
| dispersion (Aquacoat ® | of about 11-12% by |
| dispersion) | weight of the core |
| Dibutyl sebacate (20% by | |
| weight of solids content | |
| in the aqueous dispersion) | |
| Composition for covering | |
| the orifice | |
| Eudragit S 100 | 73.53 |
| Triethyl citrate | 22.06 |
| Tween 20 | 4.41 |
| Color | q.s. |

Mesalamine was mixed with the intragranular ingredients listed above, and the mixture was granulated using water as the granulating agent. The granules thus obtained were mixed with the extragranular ingredients and compressed to obtain a core. This core was then coated with the coating composition mentioned in Table 14 above to a weight gain of 11-12%. An orifice was then drilled in the core. The orifice was coated with a coating solution comprising Eudragit S 100, triethyl citrate and Tween 20. The layer or band or patch covering the orifice dissolves or erodes in the alkaline pH available in the right side of the colon. This ensures release of the mesalamine in the right side of the colon.

### EXAMPLE 9

The coating solutions that may be used to coat the orifice of the programmed drug delivery system of the present invention are exemplified below—

### EXAMPLE A

| Ingredients | Quantity (% w/w) |
|---|---|
| Aqoat AS HF (Hydroxypropyl methyl | 73.53 |
| cellulose acetate succinate, 7% w/w | |
| solution in suitable solvent) | |
| Triethyl citrate | 22.06 |
| Tween 20 | 4.41 |
| Color | q.s. |

### EXAMPLE B

| Ingredients | Quantity (% w/w) |
|---|---|
| Eudragit S 100 (15% w/w | 73.53 |
| solution in suitable solvent) | |
| Triethyl citrate | 22.06 |
| Tween 20 | 4.41 |
| Color | q.s. |

### EXAMPLE C

| Ingredients | Quantity (% w/w) |
|---|---|
| Hydroxypropyl methylcellulose phthalate | 86.96 |
| (HPMCP HP-50 and HP-55, 5.3% solution in | |
| suitable solvent) | |
| Diethyl phthalate | 13.04 |
| Color | q.s. |

### EXAMPLE D

| Ingredients | Quantity (% w/w) |
|---|---|
| Hydroxypropyl methylcellulose | ˜25.0 |
| Ethyl cellulose | ˜75.0 |
| Color | q.s. |

While the invention has been described by reference to specific embodiments, this was done for purposes of illustration only and should not be construed to limit the spirit or the scope of the invention.

1. A programmed drug delivery system comprising:
(f) a core composition comprising one or more beneficial agents and pharmaceutically acceptable excipients, wherein at least one excipient swells when exposed to an aqueous environment,
(g) a coat surrounding the core composition, wherein the coat is impermeable to the beneficial agent and other core components, but may be permeable or impermeable to water,
(h) a passageway in the coat,
(i) a composition applied so as to cover the passageway, and
(j) optionally, an immediate release composition comprising the same or different beneficial agent.

2. A programmed drug delivery system as claimed in claim 1 wherein the passageway is covered with a polymer composition comprising a polymer selected from the group consisting of water soluble polymer, water swellable polymer, pH-dependent polymer and a mixture thereof

3. A programmed drug delivery system as claimed in claim 1 wherein the beneficial agent is selected from agents that are susceptible to the gastric environment.

4. A programmed drug delivery system as claimed in claim 1 wherein the beneficial agent is selected from agents that are targeted to the intestine for local action.

5. A programmed drug delivery system as claimed in claim 1 wherein the beneficial agent is selected from agents that cause bleeding or irritation of the gastric mucosa.

6. A programmed drug delivery system as claimed in claim 1 wherein the composition used to cover the passageway is such that it releases the contents of the core at a predetermined time after oral administration.

7. A programmed drug delivery system as claimed in claim 1 wherein the composition used to cover the passageway is such that it releases the contents of the core at a predetermined location in the gastrointestinal tract after oral administration.

8. A programmed drug delivery system as claimed in claim 1, wherein the system provides delayed release of the beneficial agent.

9. A programmed drug delivery system as claimed in claim 1 wherein the system provides timed release of the beneficial agent.

10. A programmed drug delivery system as claimed in claim 1 wherein the system provides pulsatile delivery of the beneficial agent.

11. A programmed drug delivery system as claimed in claim 1 wherein the system provides controlled release of the beneficial agent.

12. A programmed drug delivery system as claimed in claim 1 wherein the system provides an immediate release of a beneficial agent followed by a delayed controlled release of the same or different beneficial agent, the delay being dependent on gastric emptying time.

13. A programmed drug delivery system as claimed in claim 1 wherein the system provides an immediate release of a beneficial agent followed by a timed controlled release of the same or different beneficial agent, the delay being independent of gastric emptying time.

14. A programmed drug delivery system as claimed in claim 1 wherein the system provides an immediate release of a beneficial agent followed by a delayed release of the same or different beneficial agent in a conventional manner, the delay being dependent on gastric emptying time.

15. A programmed drug delivery system as claimed in claim 1, wherein the system provides an immediate release of a beneficial agent followed by delayed release of another beneficial agent, the delay being independent of gastric emptying time.

16. A programmed drug delivery system as claimed in claim 1 wherein the system provides delivery of the beneficial agent to a targeted site.

17. A programmed drug delivery system as claimed in claim 16 wherein the delivery is in the form of a pulse.

18. A programmed drug delivery system as claimed in claim 16 wherein the beneficial agent is delivered in a rate controlled manner.
